(19) United States
(12) Patent Application Publication
(10) Pub. No.: US 2009/0312644 A1
(43) Pub. Date: Dec. 17, 2009

(54) VITAL TISSUE DISCRIMINATION DEVICE AND METHOD

(75) Inventors: Yukio KOSUGI, Kanagawa (JP); Hamed AKBARI, Kanagawa (JP); Kazuyuki KOJIMA, Tokyo (JP); Tatsuhiko SAITOH, Kanagawa (JP); Masato TANAKA, Kanagawa (JP)

Correspondence Address: VENABLE LLP, P.O. BOX 34385, WASHINGTON, DC, 20043-9998, US

(73) Assignees: Sumitomo Electric Industries, Ltd, Osaka-shi (JP); Tokyo Institute of Technology, Tokyo (JP); Tokyo Medical and Dental University, Tokyo (JP)

(21) Appl. No.: 12/481,912

(22) Filed: Jun. 10, 2009

(30) Foreign Application Priority Data
Jun. 11, 2008 (JP) 2008-152420

(51) Int. Cl. A61B 6/00

(52) U.S. Cl. 600/473

(57) ABSTRACT

A vital tissue discrimination device, which includes infrared spectrum acquiring section (1, 2, 3, 4, 5, 6, 8, 9, 10, 12, 13 and 14) for acquiring infrared spectrum information from a vital tissue and an operating section (11) for discriminating normal from abnormal of the vital tissue based on the infrared spectrum information obtained by the infrared spectrum acquiring section, wherein the operating section (11) calculates a micro-cycle fluctuation component of the infrared spectrum information and discriminates normal from abnormal of the vital tissue based on the micro-cycle fluctuation component of the infrared spectrum information.

### BACKGROUND OF THE INVENTION

This invention relates to a vital tissue discrimination device and a method, for identifying whether a vital tissue (in vivo, in vitro) is a normal tissue or an abnormal tissue (a tumor or similar pathology) based on infrared spectrum information.

As a conventional technique for discriminating normal from abnormal vital tissues based on spectral information, an attempt to discriminate normal tissue from malignant tumor based on hyperspectral endoscopic images has been made by M. E. Martin et al. (Non-patent Reference 1). However, the method of Non-patent Reference 1 cannot find a significant difference by the spectral change in reflectance alone, because it is based on the observation of visible light region alone and there is no means for introducing illumination light having necessary intensity for hyperspectral observation safely into the body. Thus, an indirect technique for injecting a fluorescent material, porphyrin, which is specifically absorbed by tumors and observing the thereby generated fluorescence has been employed, and its efficiency has been shown by animal tests. However, since porphyrin has photo-toxicity, its high concentration administration to the human body accompanies a danger. In Non-patent Reference 2, a diagnostic index of a diabetes mellitus-derived disease is calculated from visible region hyperspectral images of the living body, but since diagnosis of microcirculation is carried out in this method by actualizing absorption characteristics of different oxidized hemoglobin and reduced hemoglobin at about 600 nm to 800 nm, this cannot evaluate malignancy of the tissue itself.

### [Non-Patent Reference 1]

M. E. Martin et al.: “Development of an Advanced Hyperspectral Imaging (HSI) System with Application for Cancer Detection”, Annals of Biomedical Engineering, Vol. 34, No. 6, pp. 1061-1068 (2006)

### [Non-Patent Reference 2]

L. Khaodhiar et al.: “The Use of Medical Hyperspectral Technology to Evaluate Microcirculatory Changes in Diabetic Foot Ulcers and to Predict Clinical Outcomes”, Diabetes Care, Vol. 30, No. 4, pp. 903-910 (2007)

As described in the above papers, visible light spectrum information is used as spectrum information by the conventional tumor discrimination techniques, so that it is apt to undergo influence of the blood (particularly hemoglobin) and it is difficult to obtain spectrum information on vital tissues themselves. In order to facilitate acquirement of information on vital tissues themselves, a fluorescent material is administered to patients, but there is a possibility that the fluorescent material is not desirable for the human body. In addition, since it is necessary to administer the fluorescent material to patients in advance, burden on the patients is large.

### SUMMARY OF THE INVENTION

The invention aims at solving the above-mentioned problems and thereby providing a vital tissue discrimination device and a method, which can discriminate normal from abnormal (tumor) of vital tissues based on spectrum information without using a fluorescent material.

In order to achieve the above-mentioned object, the invention has the following means.

According to the first aspect of the invention, there is provided a vital tissue discrimination device, including:

an infrared spectrum acquiring section for acquiring infrared spectrum information from a vital tissue, and an operating section (operating unit 11) for discriminating normal from abnormal of the vital tissue based on the infrared spectrum information obtained by the infrared spectrum acquiring section, wherein

the operating section calculates a micro-cycle fluctuation component of the infrared spectrum information, and discriminates normal from abnormal of the vital tissue based on the micro-cycle fluctuation component of the infrared spectrum information.

According to the first aspect of the invention, whether a vital tissue is normal or abnormal (a tumor or similar pathology) can be identified based on the micro-cycle fluctuation component of the infrared spectrum information. Since an infrared light, particularly an infrared light of 1,000 nm or more in wavelength, is used, it is hard to undergo influence of absorption of hemoglobin in the blood so that spectrum information on the tissue itself can be obtained. The present inventors have actually measured the infrared spectrum information on a normal tissue and an abnormal tissue (tumor, cancer tissue or similar pathology) and compared the results, and found as a result that a micro-cycle fluctuation component of spectrum which is not present in the normal tissue is present in the abnormal tissue. As shown by an example of the graph of FIG. 1 on the actually measured infrared spectrum information on a normal tissue and an abnormal tissue (gastric cancer tissue), it can be understood that there is a minute fluctuation (micro-cycle fluctuation component) at a wavelength of about 1,200 nm to 1,320 nm, which is not present in the normal tissue. The invention discriminates an abnormal tissue from a normal tissue based on this minute spectral fluctuation (micro-cycle fluctuation component) In this connection, though attention is paid to a micro-cycle fluctuation component at a wavelength of about 1,200 nm to 1,320 nm in the example, it goes without saying that a micro-cycle fluctuation component of not only this wavelength region but of other infrared wavelength region may also be employed. For example, it can be seen that a similar micro-cycle fluctuation component is present at a wavelength of about 1,500 nm to 1,600 nm.

Besides, in the present invention, the micro-cycle fluctuation component of the infrared spectrum information indicates a spectrum fluctuation in a wavelength period of 30 nm or less.

According to the second aspect of the invention, there is provided the vital tissue discrimination device according to the first aspect, wherein

the infrared spectrum acquiring section includes:

an infrared light source which can sweep the central wavelength at the infrared region, and

an infrared light detecting section having a sensitivity of the infrared region, wherein

the infrared spectrum information is obtained by applying the infrared light emitted from the infrared light source to the vital tissue and detecting the measuring light reflects, penetrates or scatters in the vital tissue by the infrared light detecting section.

According to the second aspect of the invention, since an infrared light of a specific wavelength is applied to a vital tissue by sweeping the central wavelength at the infrared light source side, only an infrared light having a limited wavelength is applied to the vital tissue so that excess light energy is not applied to the vital tissue. Thus, thermal influence on the vital tissue by irradiation light is little in comparison with the spectral operation at the side of light detecting section.

According to the third aspect of the invention, there is provided the vital tissue discrimination device according to the second aspect, wherein

the infrared light detecting section is an image picking-up section which can detect infrared light, and

the infrared spectrum acquiring section acquires the infrared spectrum information for each picture element of the image picked-up by the image picking-up section.

According to the third aspect of the invention, infrared spectrum information can be obtained for each picture element of a two-dimensional image by the image picking-up section, so that abnormal tissues can be identified at further high accuracy. Since spectrum information is obtained by sweeping the wavelength at the side of infrared light source, a spectral section is not necessary for the image picking-up section so that a general infrared image picking-up section (CCD or the like) can be used.

According to the forth aspect of the invention, there is provided the vital tissue discrimination device according to any one of the first to the third aspects, wherein

the infrared spectrum information is infrared spectrum information within a wavelength range of from 1,200 to 1,320 nm.

According to the forth aspect of the invention, it is found based on a test carried out by the present inventors that a difference in the micro-cycle fluctuation component of spectrum information between normal tissue and abnormal tissue and a difference in the spectral pattern between normal tissue and abnormal tissue are particularly significant within this wavelength region.

According to the fifth aspect of the invention, there is provided the vital tissue discrimination device according to any one of the first to forth aspects, wherein

the operating section calculates the micro-cycle fluctuation component by wavelet conversion of the infrared spectrum information, and thereby discriminates normal from abnormal of the vital tissue.

According to the sixth aspect of the invention, there is provided the vital tissue discrimination device according to any one of the first to fifth aspects, wherein

the operating section further includes a spectral pattern comparing part which compares spectral pattern of the infrared spectrum information with a standard spectral pattern of a normal tissue memorized in advance, and

the operating section discriminates normal from abnormal of the vital tissue based on the result of comparing micro-cycle fluctuation component of the infrared spectrum information with the spectral pattern comparing part.

According to the sixth aspect of the invention, it is found based on a test carried out by the present inventors that the spectral pattern of infrared spectrum information is different between normal tissue and abnormal tissue. By comparing the spectral patterns, in addition to the comparison of micro-cycle fluctuation components of the infrared spectrum, it is able to discriminate normal from abnormal vital tissues at further high accuracy.

According to the seventh aspect of the invention, there is provided the vital tissue discrimination device according to the sixth aspect, wherein

the spectral pattern comparing part compares the spectral pattern with the standard spectral pattern based on the correlation coefficient.

According to the eighth aspect of the invention, there is provided the vital tissue discrimination device according to any one of the first to seventh aspects, wherein

the operating section discriminates normal from abnormal of the vital tissue using a learning classification type algorithm.

According to the ninth aspect of the invention, there is provided the vital tissue discrimination device according to anyone of the first to eighth aspects, wherein

the vital tissue is a living, that is, in vivo tissue.

According to the tenth aspect of the invention, there is provided the vital tissue discrimination device according to any one of the first to ninth aspects, wherein

the infrared spectrum acquiring section acquires infrared spectrum information on a vital tissue in the living body by an endoscope.

According to the eleventh aspect of the invention, there is provided the vital tissue discrimination device according to anyone of the first to eighth aspects, wherein

the vital tissue is a tissue cut out from the living body, that is, an in vitro tissue.

According to the twelfth aspect of the invention, there is provided the vital tissue discrimination device according to any one of the first to eleventh aspects, wherein

the vital tissue identified as abnormal by the operating section is a tumor of digestive system.

According to the twelfth aspect of the invention, it is found based on a test carried out by the present inventors that the discrimination of the invention of normal from abnormal vital tissues is particularly effective for the identification of digestive system tumors. It is possible to inspect digestive system tumors, for example, by an endoscope.

According to the thirteenth aspect of the invention, there is provided a vital tissue discrimination method for discriminating normal from abnormal of the vital tissue based on the infrared spectrum information from the vital tissue obtained by an infrared spectrum acquiring section, the method including:

a step for calculating a micro-cycle fluctuation component of the infrared spectrum information, and

a step for discriminating normal from abnormal of the vital tissue based on the micro-cycle fluctuation component of the infrared spectrum information.

According to the fourteenth aspect of the invention, there is provided a vital tissue discrimination method for discriminating normal from abnormal of the vital tissue based on the infrared spectrum information from the vital tissue obtained by an infrared spectrum acquiring section, the method including:

a step for calculating a micro-cycle fluctuation component of the infrared spectrum information,

a step for comparing spectral patterns, which compares spectral pattern of the infrared spectrum information with a standard spectral pattern of a normal tissue memorized in advance, and

a step for discriminating normal from abnormal of the vital tissue based on the result of comparing micro-cycle fluctuation component of the infrared spectrum information with the spectral pattern comparing step.

According to the invention, normal and abnormal (tumor) of vital tissues can be discriminated based on spectrum information without using a fluorescent material. Since spectrum information of infrared light is used as the spectrum information, it is hard to undergo influence of the blood (particularly hemoglobin) and it is easy to obtain the spectrum information on vital tissues themselves without using a fluorescent material. Since any fluorescent material is not used, influence on the human body is less and the inspection also becomes convenient.

### BRIEF DESCRIPTION OF THE DRAWINGS

FIG. 1 is a graph showing that the reflectance spectral pattern in infrared region shows different characteristics in a normal tissue and a malignant tumor tissue.

FIG. 2 is a histogram showing a result in which dispersion value of high frequency component alone is evaluated on a large number of picture elements by wavelet conversion.

FIG. 3 is a graph showing an example of the wavelet conversion of a normal tissue (left side) and a tumor tissue (right side).

FIG. 4 is an image of a tissue in the stomach in which dispersion values at 1,200 to 1,320 nm of the wavelet conversion D1 component are made into an image.

FIG. 5 is a graph showing construction of a vital tissue discrimination device for detecting a malignant region based on the periodical change in reflection brightness (dispersion value) at the time of perturbation illumination.

FIG. 6 is a graph displaying an area (a tumor region) having a low correlation value with the spectrum of normal tissue, based on the correlation coefficient R.

FIGS. 7A and 7B are a classification image in which a hyperspectral image of a gastric cancer inner wall is classified into a tumor region (a gray islet shape part) and a normal tissue by a supervised classification, and a usual color image.

FIG. 8 is a graph in which a stomach tissue containing a tumor is sliced and a part judged as the tumor based on the situation in the tissue is marked, and a part judged as the tumor region by SVM classification based on a hyperspectral image before carrying out the slicing operation is shown by a bar (the lower step) The actual tumor region is shown by a bar on the upper step.

### DETAILED DESCRIPTION OF THE PREFERRED EMBODIMENTS

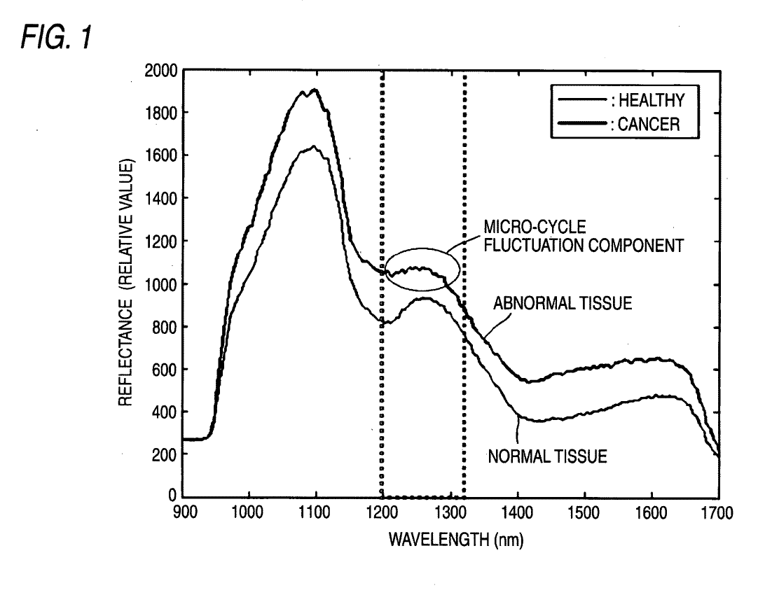

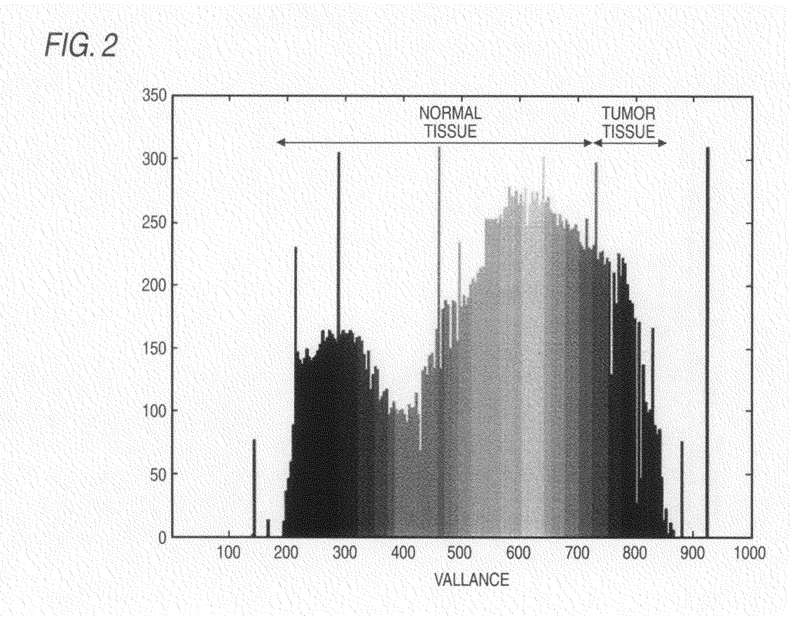

The invention is based on the knowledge that the reflectance spectral pattern at the infrared region is different between a normal cell and a malignant tumor tissue, and as shown in FIG. 1, the spectral distribution curve, particularly within the range of from 1,200 to 1,320 nm, is relatively smooth in the normal tissue, while minute fluctuation is significantly found in the tumor tissue. In order to quantify this fluctuation, dispersion value of high frequency component alone is evaluated on a large number of picture elements and expressed as a histogram in FIG. 2, showing that the dispersion value in the tumor tissue is larger than the normal range.

In addition, when two or more samples are compared, patterns of the reflectance spectrum of normal tissues at around 1,200 to 1,320 nm are almost identical, while a consistent tendency is not found in the large swell (low frequency component) on the tumor tissue but a minute fluctuation (high frequency component) for each channel is significantly observed in all of the tumor samples. A strong intensity illumination is generally required for hyperspectral imaging, but in the case of endoscopic inspection, it is better to limit the light source side wavelength to a range necessary for the observation in order to prevent damage on the tissue. For example, in the case of a hyperspectral endoscopic device in which a wavelength changeable filter is inserted into the light-intercepting side, described in a reference “J. Zuzak et al,: Characterization of a Near-Infrared Laparoscopic Hyperspectral Imaging System for Minimally Invasive Surgery, Analytical Chemistry, Vol. 79, No. 12, pp. 4709-4715, (2007)”, excessive energy is applied to the tissue from the light source.

Next, an example of the detection of a tumor tissue region according to the invention, in which the micro-cycle fluctuation component of infrared spectrum information is detected using wavelet conversion, is described.

Firstly, a reflection spectrum curve S possessed by each pixel is subjected to wavelet conversion in accordance with the following formula and developed into a high region component D1, a medium region component D2, a low region component D3 and a remainder A3. In this connection, regarding the base, Daubechies-3 wavelet function as one of the orthogonal wavelets is used as the wavelet.

$$\begin{matrix}S = {A}_{3} + {D}_{3} \\ = {A}_{2} + {D}_{2} + {D}_{1} \\ = {A}_{3} + {D}_{3} + {D}_{2} + {D}_{1}\end{matrix}$$

$${x}_{a} ( t ) = {\int }_{R} {WT}_{ψ} \{ x \} ( a , b ) \cdot {ψ}_{a , b} ( t ) d b$$

$${WT}_{ψ} \{ x \} ( a , b ) = 〈 x , {ψ}_{a , b} 〉 = {\int }_{R} x ( t ) \overline{{ψ}_{a , b} ( t )} d t \\ \text{?} \\ x ( t ) = {\sum }_{\text{?} ∈ Z} {\sum }_{\text{?} ∈ Z} 〈 x , {ψ}_{m , n} 〉 \cdot {ψ}_{m} \text{?}$$

$$\text{?} \text{indicates text missing or illegible when filed}$$

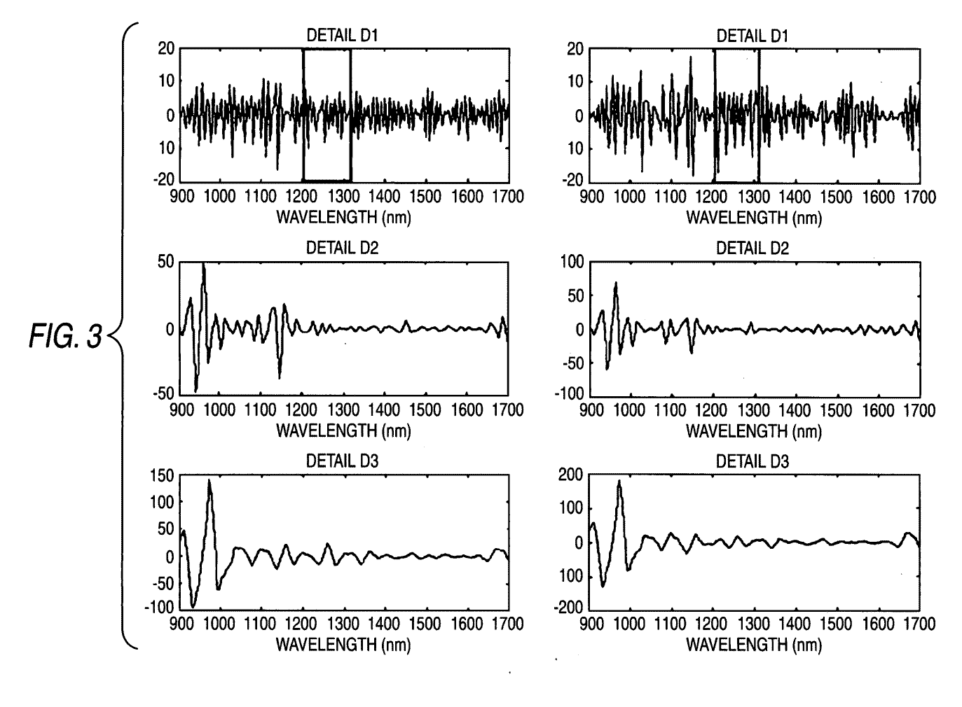

FIG. 3 is a set of graphs showing an example of the wavelet conversion of a normal tissue (left side drawing) and a tumor tissue (right side drawing), representing the components at respective irradiation wavelengths of about 17 nm in cycle as the high region component D1, and 35 nm as the medium region component D2 and 70 nm as the low region component D3, when the irradiation is carried out within a wavelength region of from 900 nm to 1,700 nm.

As shown in the same drawings, when results of the wavelet conversion are analyzed, it can be understood that the D1 which represents changes at the high region becomes high amplitude of vibration in the tumor (right side drawing) in comparison with the normal (left side drawing), particularly within the range of from 1,200 to 1,320 nm.

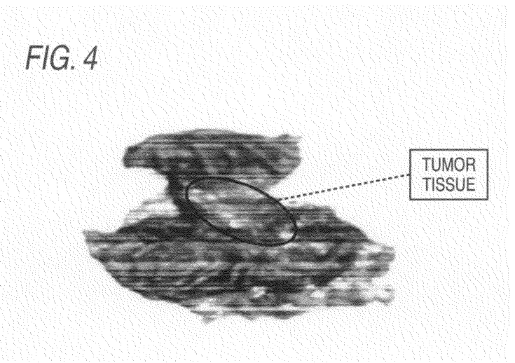

FIG. 4 is an image of a tissue in the stomach in which dispersion values at 1,200 to 1,320 nm of the wavelet conversion D1 component are made into an image.

As shown in the same drawing, the whitish part is a part having large dispersion and represents a tumor tissue.

In this connection, though an example of detecting fluctuation of micro-cycle of infrared spectrum using wavelet conversion is described, the means for detecting fluctuation of micro-cycle is not limited to the wavelet conversion and other means may also be used.

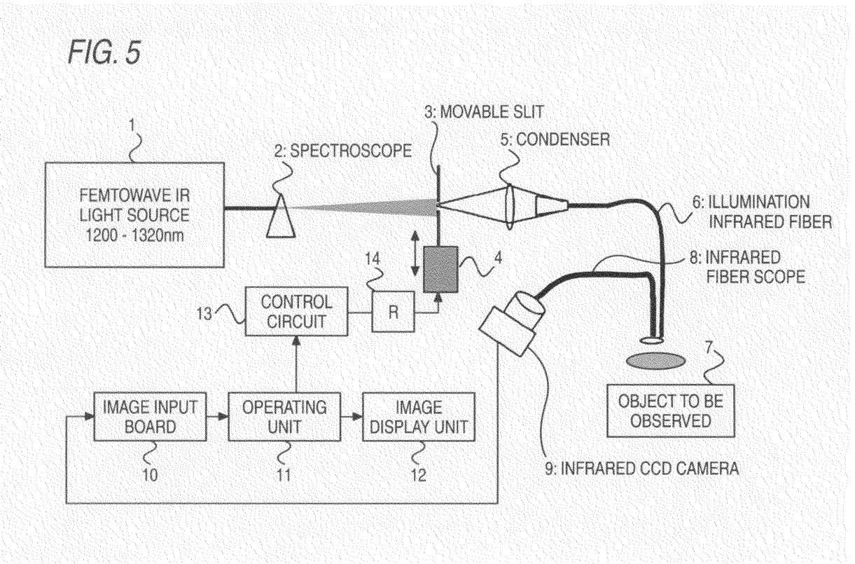

Next, a vital tissue discrimination device for detecting a malignant region based on the periodical change in reflection brightness (dispersion value) at the time of perturbation illumination is described using FIG. 5.

In this device, central wavelength of a narrow band illumination light having a band width of 5 nm is changed within a range of from 1,200 nm to 1,320, and roughness of the reflection spectrum curve of a tissue is evaluated from the second power of the image difference between continuous images obtained at 5 nm intervals and converted into an image.

In the drawing, a femtowave infrared light source 1 is generally called SC light source. When an infrared monochromatic laser of femtosecond is injected into a highly non-linier optical fiber, the monochromatic laser light is converted into a broad band white light by a non-linear optical effect which is called self phase modulation. In this case, a white light of 1,100 to 2,500 nm in wavelength is obtained by carrying out wavelength conversion by the non-linear optical fiber using a femtosecond laser of 1,550 nm in wavelength as the seed light source. The light generated by the femtowave infrared light source 1 is developed on the surface of a movable slit 3 by a spectroscope 2. The movable slit 3 is driven to a chopping wave form by a piezoelectric actuator 4, and a light within the range of from 1,205 to 1,315 nm in central wavelength, slipping off by a half band width from the continuous spectra 1,200 to 1,320 nm, is taken out and introduced into an illumination infrared fiber 6 by a condenser 5. The image of an object 7 to be observed illuminated by this light is converted into an electric signal by an infrared CCD camera 9 via an infrared fiber scope 8 consisting of an infrared multi-core fiber and an object glass and introduced into an operating unit 11 via an image input board 10.

In the operating unit 11, the image difference between adjacent frames is treated by the second power for each pixel, and the results are averaged for the past 45 frames and displayed on an image displaying device 12. In this connection, synchronously with the image uptake of the image input board 10, a clock signal is fed into a control circuit 13 of the piezoelectric actuator 4 so that a 5 nm wavelength change is obtained for each image input. That is, a symmetric chopping wave of 0.75 second in cycle is output from the control circuit 13, and its amplitude is regulated by a variable attenuator R (14) in such a manner that the output light central wavelength from the movable slit 3 becomes 1,205 to 1,315 nm.

According to this device, since it is possible to set the illumination side waveband width sufficiently narrowly, the irradiation energy which is given to the object 7 to be observed can be suppressed to a sufficiently low level so that thermal influence on the object to be observed can be sharply reduced in comparison with the hyperspectral endoscope which restricts the light-interception side waveband. In addition, since the wavelength difference is treated as image information, image buffer and operation mechanism can be simplified in comparison with the generally used hyperspectral image treating devices, so that observation with superior real time ability can be realized. In this connection, the image acquirement is set to 1/60 seconds per 1 frame in this example, but it is possible to further improve the real time ability by using a high speed camera or changing the averaging operation of difference quantities to exponential load average.

In this connection, the infrared spectrum acquiring section of the invention corresponds to the femtowave infrared light source 1, spectroscope 2, movable slit 3, piezoelectric actuator 4, condenser 5, illumination infrared fiber 6, infrared fiber scope 8, infrared CCD camera 9, image input board 10, control circuit 13 and variable attenuator 14, the operating section corresponds to the operating unit 11, the infrared light source corresponds to the femtowave infrared light source 1, spectroscope 2, movable slit 3, piezoelectric actuator 4, condenser 5 and illumination infrared fiber 6, and the infrared light detecting section corresponds to the infrared fiber scope 8 and infrared CCD camera 9.

Next, the detection of a normal tissue and an abnormal tissue based on the difference in spectral pattern, in the invention, is described. According to the invention, the detection of a normal tissue and an abnormal tissue based on the difference in spectral pattern, described in the following, can be improved to further high accuracy by combining it with the detection based on the micro-cycle fluctuation of infrared spectrum information.

Firstly, when objective reflectance series of inspection normal tissue and abnormal tissue are regarded as fᵢ (i=1 . . . N), and standard reflectance pattern of normal cell as gᵢ (i=1 . . . N), based on the spectral characteristics of within the range of from 1,200 nm to 1,320 nm, for example, as is known by “Kumagai, Kosugi et al.: “Judgment of platelet aggregation ability by ADP using a pattern matching method (written in Japanese)”, Iyo Denshi to Seitai Kogaku (Medical Electronics and Biological Engineering), Vol. 24, No. 5, pp. 321-326 (1986)” and the like, it is possible to use them as a measure of the difference from normal using the following correlation coefficient R (−1<R<1). In this case, each subscript i is an index which represents observation waveband, and i=1 corresponds to 1,200 nm, and i=N to 1,320 nm.

$$R = {\sum }_{i = 1}^{N} \frac{( {f}_{1} - {f}_{0} ) ( {g}_{1} - {g}_{0} )}{{( {S}_{1} {S}_{2} )}^{1 / 2}}$$

wherein

$${S}_{1} = {\sum }_{i = 1}^{N} {( {f}_{i} - {f}_{o} )}^{2} , {S}_{2} = {\sum }_{i = 1}^{N} {( {g}_{i} - {g}_{0} )}^{2} , {f}_{0} = {\sum }_{i = 1}^{N} {f}_{t} , {g}_{0} = {\sum }_{i = 1}^{N} {g}_{i}$$

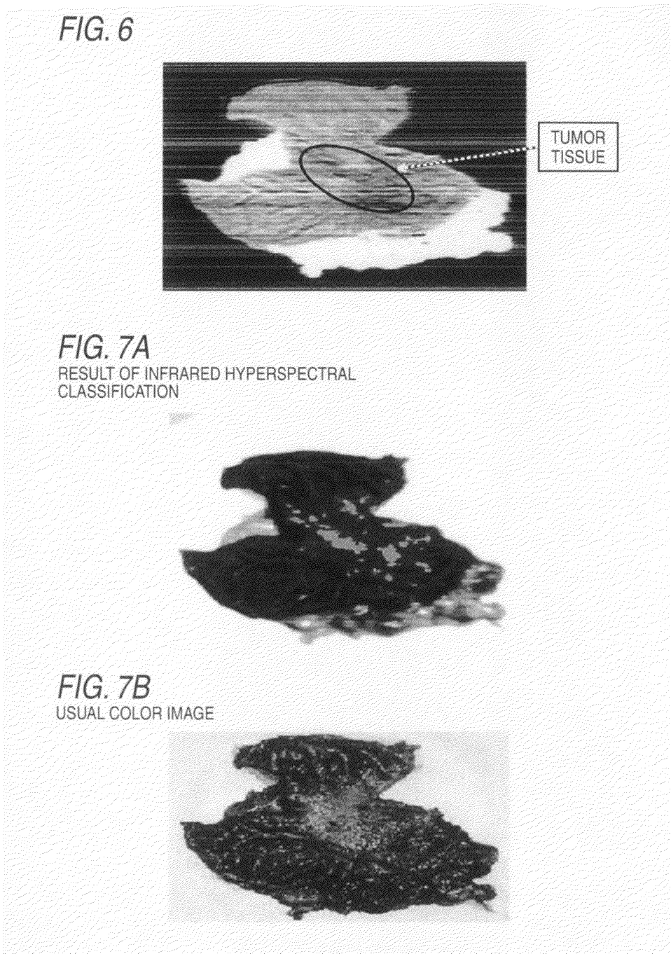

FIG. 6 is a graph displaying an area (a tumor region) having low correlation value with the spectrum of normal tissue, based on the above-mentioned correlation coefficient R.

Next, there is shown a result in which infrared spectral characteristics are classified for each picture element by a multilayer perceptron or the like neural network or a support vector machine (SVM) or the like learning type sorter, which uses normal samples and tumor samples as the training data. In a supplementary test of SVM, it results in a poor result than the wavelet method when the wavelength is limited to this range.

FIG. 7A is an image showing a result in which a hyperspectral image of a gastric cancer inner wall is classified into a tumor region (a gray islet shape part) and a normal tissue by a supervised classification, and in comparison with the usual color image shown in FIG. 7B, the tumor region (green) and normal tissue are distinctively distinguished. In addition, the above treatment results are compared with pathologic inspection data.

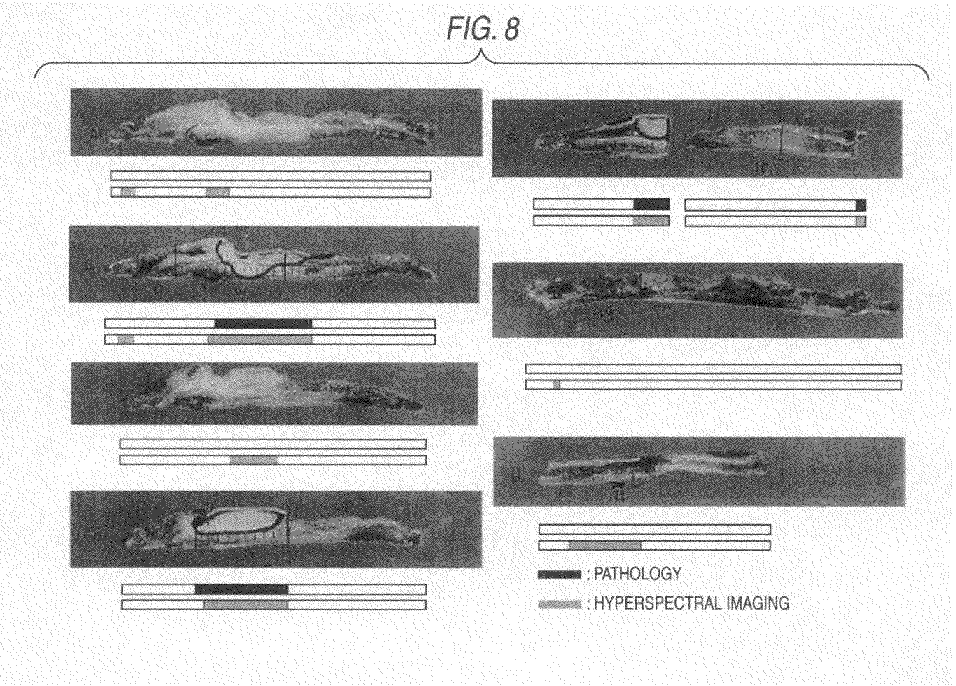

FIG. 8 is a graph in which a stomach tissue containing a tumor is sliced and a part judged as the tumor based on the situation in the tissue is marked, and contrary to this, a part judged as the tumor region by SVM classification based on a hyperspectral image before carrying out the slicing operation is shown by a bar (the lower step). (The actual tumor region is shown by a bar on the upper step.) Though a slight difference can be seen, it is possible to grasp the region judged as a tumor by a pathologic inspection almost accurately by a spectral inspection from the surface.

What is claimed is:

1. A vital tissue discrimination device, comprising:
an infrared spectrum acquiring section for acquiring infrared spectrum information from a vital tissue, and
an operating section for discriminating normal from abnormal of the vital tissue based on the infrared spectrum information obtained by the infrared spectrum acquiring section, wherein
the operating section calculates a micro-cycle fluctuation component of the infrared spectrum information, and discriminates normal from abnormal of the vital tissue based on the micro-cycle fluctuation component of the infrared spectrum information.

2. The vital tissue discrimination device according to claim 1, wherein
the infrared spectrum acquiring section comprises:
an infrared light source which can sweep the central wavelength at the infrared region, and
an infrared light detecting section having a sensitivity of the infrared region, wherein
the infrared spectrum information is obtained by applying the infrared light emitted from the infrared light source to the vital tissue and detecting the measuring light which reflects, penetrates or scatters in the vital tissue by the infrared light detecting section.

3. The vital tissue discrimination device according to claim 2, wherein
the infrared light detecting section is an image picking-up section which can detect infrared light, and
the infrared spectrum acquiring section acquires the infrared spectrum information for each picture element of the image picked-up by the image picking-up section.

4. The vital tissue discrimination device according to claim 1, wherein
the infrared spectrum information is infrared spectrum information within a wavelength range of from 1,200 to 1,320 nm.

5. The vital tissue discrimination device according to claim 1, wherein
the operating section calculates the micro-cycle fluctuation component by wavelet conversion of the infrared spectrum information.

6. The vital tissue discrimination device according to claim 1, wherein
the operating section further comprises a spectral pattern comparing part which compares spectral pattern of the infrared spectrum information with a standard spectral pattern of a normal tissue memorized in advance, and
the operating section discriminates normal from abnormal of the vital tissue based on the result of comparing micro-cycle fluctuation component of the infrared spectrum information with the spectral pattern comparing part.

7. The vital tissue discrimination device according to claim 6, wherein
the spectral pattern comparing part compares the spectral pattern with the standard spectral pattern based on the correlation coefficient.

8. The vital tissue discrimination device according to claim 1, wherein
the operating section discriminates normal from abnormal of the vital tissue using a learning classification type algorithm.

9. The vital tissue discrimination device according to claim 1, wherein
the vital tissue is a living, that is, in vivo tissue.

10. The vital tissue discrimination device according to claim 1, wherein
the infrared spectrum acquiring section acquires infrared spectrum information on a vital tissue in the living body by an endoscope.

11. The vital tissue discrimination device according to claim 1, wherein
the vital tissue is a tissue cut out from the living body, that is, an in vitro tissue.

12. The vital tissue discrimination device according to claim 1, wherein
the vital tissue identified as abnormal by the operating section is a tumor of digestive system.

13. A vital tissue discrimination method for discriminating normal from abnormal of the vital tissue based on the infrared spectrum information from the vital tissue obtained by an infrared spectrum acquiring section, the method comprising:
a step for calculating a micro-cycle fluctuation component of the infrared spectrum information, and
a step for discriminating normal from abnormal of the vital tissue based on the micro-cycle fluctuation component of the infrared spectrum information.

14. A vital tissue discrimination method for discriminating normal from abnormal of the vital tissue based on the infrared spectrum information from the vital tissue obtained by an infrared spectrum acquiring section, the method comprising:
a step for calculating a micro-cycle fluctuation component of the infrared spectrum information,
a step for comparing spectral patterns, which compares spectral pattern of the infrared spectrum information with a standard spectral pattern of a normal tissue memorized in advance, and
a step for discriminating normal from abnormal of the vital tissue based on the result of comparing micro-cycle fluctuation component of the infrared spectrum information with the spectral pattern comparing step.
